(19) United States
(12) Patent Application Publication
(10) Pub. No.: US 2010/0268043 A1
(43) Pub. Date: Oct. 21, 2010

(54) Device and Method for Preventing Diabetic Complications

(76) Inventors: Ofer Yodfat, Maccabim-Reut (IL); Gali Shapira, Haifa (IL)

Correspondence Address: MINTZ LEVIN COHN FERRIS GLOVSKY & POPEO, ONE FINANCIAL CENTER, BOSTON, MA, 02111, US

(21) Appl. No.: 12/741,715

(22) PCT Filed: Nov. 4, 2008
(86) PCT No.: PCT/IL2008/001443

§ 371 Date: May 6, 2010

Related U.S. Application Data
(60) Provisional application No. 61/002,400, filed on Nov. 7, 2007.

(51) Int. Cl. A61M 37/00; A61B 5/1473

(52) U.S. Cl. 600/345; 604/66

(57) ABSTRACT

Methods, systems and devices for preventing diabetic complications are provided. In some embodiments, methods, systems and devices can be implemented using at least one subcutaneously insertable element, the element having a proximal portion and a distal portion, the proximal portion coupled to the housing, the distal portion configured for subcutaneous placement within a human body; a plurality of electrodes coupled to the distal portion of the at least one subcutaneously insertable element; and, a processor adapted to correlate a signal received from the plurality of electrodes to a concentration of ketone in the human body.

### FIELD

A system, device and a method for sustained medical infusion of fluids and monitoring of bodily analytes are described. Some embodiments relate generally to a portable insulin infusion device and a method for preventing diabetes complications.

### BACKGROUND

Diabetes mellitus is a disease of major global importance, increasing in frequency at almost epidemic rates, such that the worldwide prevalence in 2006 is 170 million people and predicted to at least double over the next 10-15 years. Diabetes is characterized by a chronically raised blood glucose concentration (hyperglycemia), due to a relative or absolute lack of the hormone, insulin, which is produced by the pancreas. Within the healthy pancreas, beta cells, located in the islets of Langerhans, continuously produce and secrete insulin according to the blood glucose levels, thus maintaining near constant glucose levels in the body.

Diabetes can cause acute and chronic complications. Acute complications can include hypoglycemia and ketoacidosis. Long-term complications, for example, due to the affect on small and large blood vessels, can include eye, kidney and nerve damage and accelerated atherosclerosis, with increased rates of coronary heart disease, peripheral vascular disease and stroke accordingly.

The Diabetes Control and Complications Trial (DCCT) demonstrated that development and progression of the chronic complications of diabetes are greatly related to the degree of altered glycemia as quantified by determinations of glycosylated hemoglobin (HbA1c). [DCCT Trial, N Engl J Med 1993; 329: 977-986, UKPDS Trial, Lancet 1998; 352: 837-853. BMJ 1998; 317, (7160): 703-13 and the EDIC Trial, N Engl J Med 2005; 353, (25): 2643-53]. Hemoglobin A1c

Glycosylated hemoglobin (HbA1c) is a form of hemoglobin used primarily to identify the plasma glucose concentration over prolonged periods of time (e.g. 3 months). It is formed in a non-enzymatic pathway by hemoglobin's normal exposure to high plasma levels of glucose. A high Hb A1c represents poor glucose control. An exemplary relationship between Hb_{A1c} and blood glucose average is shown in the FIG. 1 adapted from http://en.wikipedia.org/wiki/Hemoglobin_A1c.

Lower than expected levels of Hb A1c can be seen in people who have an increased red blood cell turn-over (e.g. G6PD, sickle-cell disease, hypersplenism). Higher than expected levels can be seen in people with a lower RBC turn-over (e.g. vitamin B12 or folate deficiency). HbA1c is therefore not indicative of the blood glucose average in people with pathological red blood cells.

Currently, most diabetes patients have to attend the clinic for their HbA1c to be tested. A follow-up visit is usually required to obtain and discuss the results. A device and a method for determining glycemic condition using single test system to measure glucose and protein-bound glucose levels (e.g. HbA1c) has been described in U.S. Pat. No. 6,958,129 assigned to LifeScan.Inc.

Diabetic ketoacidosis (DKA) is a life-threatening, acute complication of diabetes that highly contributes to the morbidity and mortality of type 1 diabetes patients and affects the direct and indirect costs of healthcare. DKA results from insulin deficiency, in combination with increased levels of stress hormones that stimulate lipolysis, resulting in the production of ketone bodies (acetoacetatate, β-hydroxybutyrate (β-OHB) and acetone). Excessive production of ketone bodies lowers the pH of the blood, and causes severe dehydration and blood hyperosmolarity. DKA can be precipitated by infection, other intercurrent illnesses (e.g. surgery, trauma), psychological stress, noncompliance with insulin therapy, and pregnancy.

DKA may be preventable in patients with diabetes if the presence of ketones is detected early and treatment is initiated. Blood ketone monitoring compared with urine testing would allow more timely home interventions and thus reduce the need for emergency or hospital interventions (Diabetic Medicine, 23, 278-284).

The methods most commonly used for the measurement of ketones in clinical practice are urine dipstick tests based on the use of a nitroprusside reaction (e.g. commercially available kits such as Ketostix', Acetest') which gives a semiquantitative measure of acetoacetate, nitropruside reacts weakly with acetone but does not take β-OHB, the predominant ketone body contributing to the acidosis, into consideration. In addition, false positive results are not uncommon in dipstick tests if the user uses concomitantly certain, drugs (e.g. Captopril).

The Precision Extra™ meter (Medi-Sense/Abbott Diabetes Care, Abbott Park, Ill.) is a combined glucose and ketone sensor that produces an electrical current proportional to blood β-OHB concentration. By using a 10-μL blood sample, this hand-held sensor produces β-OHB results within 30 seconds. (DIABETES TECHNOLOGY & THERAPEUTICS 8 (6), 2006, p. 671-676).

Frequent insulin administration can be done by multiple daily injections (MDI) with syringe or by continuous subcutaneous insulin injection (CSII) carried out by insulin pumps. In recent years, ambulatory portable insulin infusion pumps have emerged as a superior alternative to multiple daily injections of insulin. These pumps, which deliver insulin at a continuous basal rate as well as in bolus volumes, were developed to liberate patients from repeated self-administered injections, and allow greater flexibility in dose administration.

Several ambulatory insulin infusion devices are currently available on the market. The first generation of such devices employ disposable syringe-type reservoir and tubes. These devices have been described in 1972, by Hobbs, in U.S. Pat. No. 2,631,847, and in 1973, by Kaminski, in U.S. Pat. No. 3,771,694, and later by Julius, in U.S. Pat. No. 4,657,486, and by Skakoon, U.S. Pat. No. 4,544,369. The main drawbacks of these devices are their large size and weight, caused by their spatial configuration and the relatively large driving mechanism associated with the syringe and the piston. These relatively bulky devices can be carried in a patient's pocket or be attached to a belt. Consequently, the fluid delivery tube becomes long, usually longer than 40 cm, to permit needle insertion in remote locations of the body. These uncomfortable bulky devices with a long tube are rejected by the majority of diabetic insulin users, since they disturb regular activities, such as sleeping and swimming for example. In addition, the long delivery tube may not be suitable for some optional remote insertion sites, like the buttocks and the extremities.

To avoid the limitations associated with the necessity of using long delivery tube, a second generation of infusion devices was proposed. This concept includes a remote controlled skin adherable device with a housing having a bottom surface adapted to contact patient's skin, a reservoir disposed within the housing, and an injection needle adapted to communicate with the reservoir. These skin adherable devices are disposed every 2-3 days similarly to available pump infusion sets. These devices were disclosed at least in U.S. Pat. No. 5,957,895 to Sage, U.S. Pat. No. 6,589,229 to Connelly, and U.S. Pat. No. 6,740,059 to Flaherty. Additional configurations of skin adherable pumps were disclosed in U.S. Pat. No. 6,723,072 to Flaherty and U.S. Pat. No. 6,485,461 to Mason. These devices also have several limitations: they are also bulky and expensive. Their high selling price is due to the high production and accessory costs; the user must discard the entire device every 2-3 days, including the relatively expensive components, such as driving mechanism and electronics.

A third generation dispensing device, as the Medingo Pump described in patent co-pending/co-owned U.S. patent application Ser. No. 11/397,115 and International Patent Application No. PCT/IL06/001276, is a miniature portable programmable fluid dispenser that has no tubing and can be attached to the patient skin. It is composed of two parts, a disposable part (DP) and a reusable part (RP). After connection of the reusable and the disposable parts, the unified dispensing unit presents a thin profile. The RP contains electronics and other relatively expensive components and the DP contains reservoir. The Medingo device comprises a remote control unit that allows data acquisition, programming, and user inputs. An improvement to a skin adherable pump that consists of the two parts is described in co-pending/co-owned U.S. Patent Application Ser. No. 60/876,679, disclosure of which is incorporated herein by reference in its entirety. In this application, a system and a method for connection and disconnection of a skin securable pump is disclosed. The method uses a cradle, which is initially adhered to the skin and then a cannula is inserted through the cradle into the body of the user. The two-part pump can be consequently connected and disconnected to and from the cradle upon patient discretion.

Another Medingo pump is described in U.S. patent application Ser. No. 11/706,606. However, this disclosure does not include a description of a pump which detects bodily ketones, and therefore, cannot make early detection and warning of impending DKA.

### SUMMARY

Methods, systems and devices for preventing diabetic complications are provided. In some embodiments, methods, systems and devices can be implemented using at least one subcutaneously insertable element, the element having a proximal portion and a distal portion, the proximal portion coupled to the housing, the distal portion configured for subcutaneous placement within a human body; a plurality of electrodes coupled to the distal portion of the at least one subcutaneously insertable element; and, a processor adapted to correlate a signal received from the plurality of electrodes to a concentration of ketone in the human body.

For example, the processor can be wirelessly connected to the plurality of electrodes. The processor can be adapted to correlate the signal received from the plurality of electrodes to a concentration of glucose in the human body. The plurality of electrodes can comprise a first electrode having a first exposed surface that can be at least partially coated with an electrochemical reagent and a second electrode having a second exposed surface that can be substantially free of the electrochemical reagent.

In some embodiments, the electrochemical reagent can comprise an enzymatic assay that can undergo a chemical reaction with analyte bodies, the chemical reaction producing a measurable voltage or current differential between the first electrode and the second electrode.

The processor can be positioned in a remote control unit. The remote control unit can comprise an HbA1c measurement device. The processor can also be configured to generate a notification of impending diabetic ketoacidosis based on the signals received from the plurality of electrodes.

The processor can be adapted for continuous monitoring of the concentration of ketone in a subcutaneous compartment of the human body. For example, the continuous monitoring can be accomplished by performing discrete measurements via at least one of a high measurement rate and short inter-measurement intervals.

The at least one subcutaneously insertable element can be configured for monitoring ketone levels in the subcutaneous tissue and another subcutaneously insertable element can be configured for continuously dispensing insulin to the subcutaneous tissue.

The concentration of ketone in the human body can be determined based on an enzymatic dehydrogenation to acetoacetate by 3-hydroxybutyrate dehydrogenase (HBDH), followed by the detection of the reduced form of nicotinamide adenine (NADH). The generated NADH can further be recycled to NAD+ by salicyclate hydroxylase (SHL).

The at least one of the plurality of electrodes can be covered by a immobilized enzyme. For example, the immobilized enzyme can be 3-hydroxybutyrate dehydrogenase (HBDH). The immobilized enzymes can also be glucose oxidase (GOX). The at least one of the plurality of electrodes can comprise different layers that can modulate the diffusion of multiple analytes in the subcutaneous tissue. The at least one subcutaneously insertable element can be configured for monitoring ketone levels and dispensing insulin into the subcutaneous tissue.

In some embodiments, the device can further comprise a pumping mechanism. The pumping mechanism can be peristaltic. The pumping mechanism can also comprise a syringe with a propelling plunger. In some embodiments, the housing can comprise a reusable part and a disposable part, the disposable part can comprise the reservoir and the reusable part can comprise at least a portion of a pumping mechanism.

In another embodiment, a method for generating a notification for a user of a ketone management device can be provided. For example, the method can comprise receiving a first value corresponding to a ketone concentration level at a first point in time; storing the first value for a subsequent retrieval; comparing the first value to an impending value; generating a notification for a user to check the ketone levels if the first value exceeds the impending value. The method can also comprise receiving a second value corresponding to a blood glucose concentration level at a second point in time; and, generating a recommendation for the user to check the ketone levels if the difference between the first value and the second value exceeds a predefined threshold.

In another embodiment, a method for determining a concentration of ketone in a human body can be provided. In some embodiments, the method can comprise inserting at least one subcutaneously insertable element into a subcutaneous compartment of a human body, the element having a proximal portion and a distal portion, the proximal portion coupled to the housing, the distal portion configured for subcutaneous placement within a human body; receiving a signal from a plurality of electrodes coupled to the at least one subcutaneously insertable element; and, by a processor, correlating the signal to a concentration of ketone in the human body.

### BRIEF DESCRIPTION OF THE DRAWINGS

FIG. 1 provides an exemplary relationship between Hb_{A1c} and blood glucose average.

FIG. 2 provides an example of a two part, skin adhered dispensing patch unit and a remote control unit.

FIG. 3 provides one example of a dispensing patch unit and a subcutaneous ISF ketone sensing apparatus.

FIG. 4 provides one example of a dispensing patch unit, a subcutaneous ISF glucose sensing apparatus, and a subcutaneous ISF ketone sensing apparatus.

FIG. 5 provides one example of a device comprising a dispensing patch unit with a subcutaneous ISF ketone sensing apparatus, and a remote control unit that contains a blood glucose monitor (glucometer) for discrete blood glucose measurements.

FIG. 6 provides one example of a device comprising a dispensing patch unit with a subcutaneous ISF ketone sensing apparatus, and a remote control unit containing a blood glucose monitor and hemoglobin A1c (HA1c) monitor.

FIG. 7 provides one example of a two part dispensing patch unit that contains a ketone sensing apparatus for continuously monitoring ketone levels in the ISF.

FIG. 8 provides one example of a two part dispensing patch unit that can continuously monitor glucose levels and ketone levels in the ISF.

FIG. 9 provides one example of a two part dispensing patch unit and detailed description of the subcutaneous ISF ketone sensing apparatus within the disposable and reusable parts.

FIG. 10 provides one example of a device that comprises a two part dispensing patch unit and a cradle unit and can continuously monitor ISF glucose and ketone levels.

FIG. 11 provides one example of a cannula insertion process using a designated inserter.

FIG. 12 provides one example of a device comprising a dispensing patch unit and a remote control unit wherein the device comprises a blood ketone monitor and a blood glucose monitor for discrete ketone and glucose measurements.

FIG. 13 provides one example of a device comprising an insulin dispensing patch unit and a remote control unit that contain a blood glucose, ketone and HbA1c monitors.

FIG. 14 provides one example of a device comprising a two part dispensing patch unit and a remote control that contain a blood glucose monitor, and a blood ketone monitor.

FIG. 15 provides some examples of a system that comprises an insulin infusion device and a continuous, subcutaneously located ISF glucose monitor and an external blood ketone meter for discrete ketone measurements.

FIG. 16 provides one example of a device that includes a dispensing patch unit composed of a reusable part and a disposable part and a remote control unit that contains a blood glucose monitor and a blood ketone monitor.

FIG. 17 provides one example of a device comprising a two part patch unit and a remote control unit that includes a blood ketone monitor and can wirelessly receive blood glucose readings from a remote monitor.

FIG. 18 provides some examples of a remote control unit that can contain blood glucose and blood ketone monitors.

FIG. 19 provides one example of a remote control unit with a screen displaying a reminder for checking ketone level in response to detection of high blood glucose levels.

FIG. 20 provides one example of a device comprising a two part patch unit with a subcutaneous ISF glucose sensing apparatus, a subcutaneous ISF ketone sensing apparatus and a display screen located on the reusable part of the patch unit.

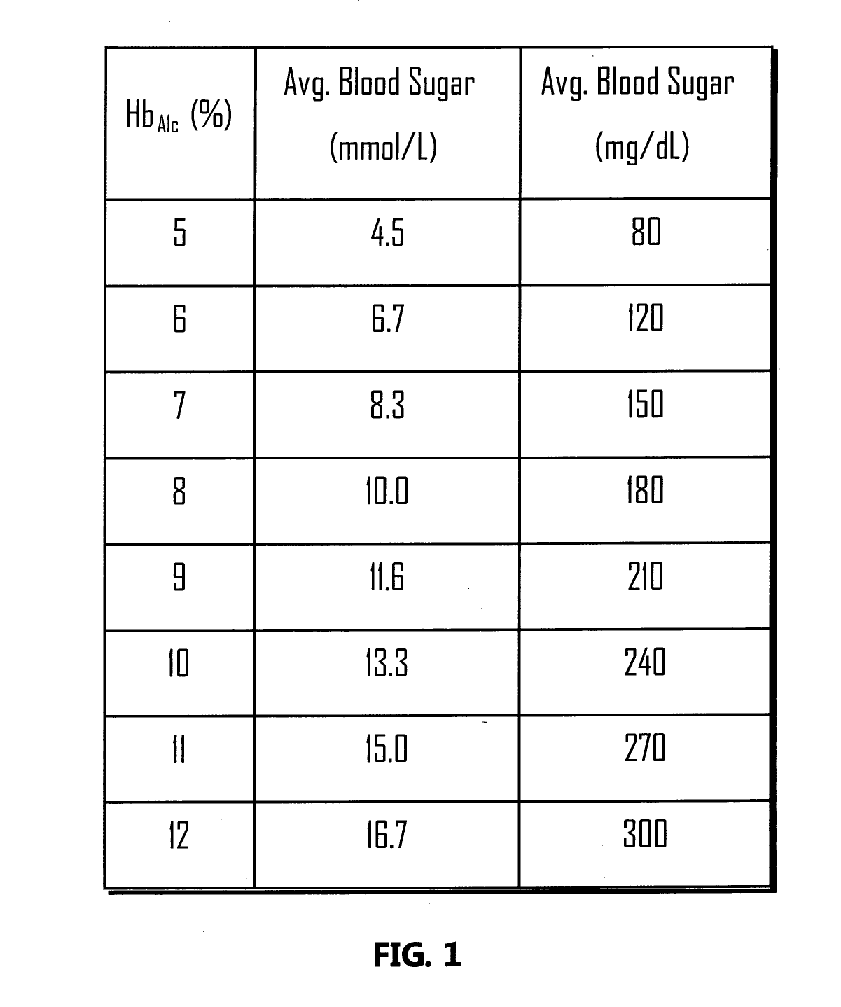

### DETAILED DESCRIPTION

Methods, systems and devices for preventing diabetic complications are provided. In some embodiments, methods, systems and devices can be implemented using at least one subcutaneously insertable element, the element having a proximal portion and a distal portion, the proximal portion coupled to the housing, the distal portion configured for subcutaneous placement within a human body; a plurality of electrodes coupled to the distal portion of the at least one subcutaneously insertable element; and, a processor adapted to correlate a signal received from the plurality of electrodes to a concentration of ketone in the human body.

In some embodiments, the ketone management device can be suitable for programming manually with the use of a operating key(s) provided at the dispensing patch unit. The dispensing patch unit can be composed of two parts—a disposable part and reusable part. The disposable part can contain a reservoir and an outlet port which can be provided with a connecting lumen and electrical connectors. The reusable part can contain electronics (PCB, processor, etc), a driving mechanism and metering portion.

A cradle unit can also be provided that adheres to the skin and allows the dispensing patch unit disconnection and reconnection to the user upon patient discretion. After attachment of the cradle unit to the skin, a cannula can be inserted into the subcutaneous tissue of the patient's body through a dedicated passageway (“well”) provided in the cradle unit. Cannula insertion can be done manually or automatically (with inserter) using a penetrating member having a sharp tip. After insertion of the cannula, the patch unit can be connected to the cradle unit.

During connection of the patch to the cradle unit, the connecting lumen provided in the patch unit can pierce a self-sealable rubber septum provided at the upper portion of the cannula, thereby establishing fluid communication between the reservoir and the cannula.

Electrodes can be attached to the outer surface of the cannula. The electrodes can be covered by immobilized enzymes (for example), which enable sensing of analytes. For example, for the detection of ISF ketone bodies, enzyme 3-hydroxybutyrate dehydrogenase (HBDH) can be used for covering the cannula, and for ISF glucose detection, the enzyme glucose oxidase (GOX) can be used. Accordingly, the electrons generated by the enzymatic reaction can be accepted by the electrodes and the resulting electrical current, which is proportional to the analyte concentration, can flow by wires to electrical connectors provided at the upper portion of the cannula. The outlet port can also be provided with connectors, and then after connection of the patch unit to the cradle, the cannula connectors come into contact with the outlet port connectors and the electrical current proceeds to the processor of the patch unit where it can be analyzed and expressed as analyte concentration (i.e. glucose and/or ketones).

Connectors can also be deployed in the well, and thus, when cannula connectors come into contact with connectors in the well, the electrical current can flow through appropriate electrical paths provided in the cradle to an additional set of connectors which would contact corresponding connectors provided in the reusable part of the patch unit.

Data processing can be carried out in the remote control unit. According to the analyzed data, appropriate alarms and alerts can be annunciated either by the remote control unit and/or by the patch unit. The processor of the patch unit can adjust the fluid delivery according to the detected analyte levels. For example, if glucose levels are high, insulin basal delivery rate will increase.

Several configurations for a device/system that include a dispensing patch unit for therapeutic fluid delivery into the body (i.e. insulin) and at the same time can monitor bodily analyte levels (i.e. ketones and glucose) can be provided:

1. An insulin dispensing patch that comprises a continuous subcutaneous glucose monitor and a continuous subcutaneous ketone monitor.

2. An insulin dispensing patch that comprises a continuous subcutaneous glucose monitor and an external blood ketone monitor for discrete ketone measurements.

3. An insulin dispensing patch that comprises an external blood glucose monitor for discrete glucose measurements and a continuous subcutaneous ketone monitor.

4. An insulin dispensing patch that comprises an external blood glucose monitor for discrete glucose measurements and an external blood ketone monitor for discrete ketone measurements.

The provided device can continuously dispense insulin to the subcutaneous (SC) tissue and monitor ketone levels in the ISF. For example, the same cannula can be used for insulin dispensing and ketone sensing. Alternatively, two cannulae could be inserted to the SC tissue—one cannula for insulin delivery and another cannula for ketone sensing.

The provided device can continuously dispenses insulin to the subcutaneous (SC) tissue, and monitors BG and ketone levels in the ISF. The same cannula can be used for insulin dispensing, glucose sensing and ketone sensing. Alternatively, glucose sensing and ketone sensing can be carried out by using a first cannula and insulin can be delivered via an independent, second cannula. Alternatively, three cannulae can be inserted in the SC tissue.

The provided device can continuously dispense insulin to the subcutaneous (SC) tissue, monitor ketone levels in the ISF, and include a glucometer for discrete blood glucose measurements. The ketone measured in the ISF can be 3-hydroxybutyrate, which can be sensed by virtue of an enzymatic dehydrogenation to acetoacetate by 3-hydroxybutyrate dehydrogenase (HBDH), followed by the detection of the reduced form of nicotinamide adenine (NADH). The suitable enzyme can be immobilized on a subcutaneously inserted electrodes to enable measurement of ISF 3-hydroxybutyrate. The generated NADH can further be recycled to NAD⁺ by salicyclate hydroxylase (SHL). The enzymes can be co-immobilized on subcutaneously inserted electrodes.

The subcutaneously inserted electrodes can comprise different layers that modulate the diffusion of certain analytes in the ISF. The infusion device can be provided with an insulin infusion patch unit and a remote control unit. A glucometer and a blood ketone-meter can be integrated in the remote control unit of the device for discrete blood glucose and blood ketones measurements.

The infusion device can also be provided with an insulin infusion patch unit composed of reusable and disposable parts. A glucometer and a blood ketone-meter can be integrated in the reusable part of the infusion patch unit of the device. A blood ketone meter can be integrated in the reusable part of the infusion patch unit of the device and a glucometer can be integrated in the remote control unit of the device. The glucometer can also be integrated in the reusable part of the infusion patch unit of the device and a blood ketone meter can be integrated in the remote control unit of the device.

According to another embodiment, an infusion device is provided which includes a patch that can dispense insulin and continuously monitor body glucose concentration levels (e.g. blood glucose, ISF glucose) and a remote control unit. A blood ketone-meter for discrete blood ketone measurements is integrated in the remote control unit of the device.

The infusion device can be provided with a patch unit that can dispense insulin and continuously monitor body glucose concentration levels (e.g. blood glucose, ISF glucose) and can be composed of a reusable and disposable parts. For example, a blood ketone-meter can be integrated in the reusable part of the infusion patch unit of the device.

In some embodiments, the same test strip can be applied to determine the blood glucose level using the glucometer and the blood-ketone level using the ketone-meter. In some embodiments, if glucose concentration levels are continuously monitored and ketones are measures discretely, the device can recommend the user to check the ketone levels if blood glucose exceeds a certain “impending value” (e.g. 200 mg/dL). Alternatively, if a rapid rise in BG occurs, the device can recommend the user to check the ketone levels. According to one such embodiment, the user can determine the impending value in the setting of the device. The impending value can be changed to comply with certain situations (e.g. during pregnancy a lower BG threshold can be required for DKA to occur due to superimposed insulin resistance, or during illness).

In another embodiment, if glucose concentration levels are measured discretely (i.e. with a glucometer) and a discrete value exceeds an “impending value”, the user can be recommended to test for ketones. The ketone meter can also check only the b-OHB levels in the blood or ISF.

The infusion device can be provided with an insulin infusion patch unit and a remote control unit. A glucometer, a blood ketone-meter, and HbA1c meter can be integrated in the remote control unit of the device. The infusion device can be provided with an insulin infusion patch unit and a remote control unit. A glucometer and HbA1c meter can be integrated in the remote control unit of the device.

According to another preferred embodiment, an infusion device is provided which continuously dispenses insulin to the subcutaneous (SC) tissue, monitors BG and ketone levels in the ISF, and can periodically measure glycosylayted hemoglobin with a HbA1c meter.

The infusion device can continuously dispense insulin to the subcutaneous (SC) tissue, monitor ketone levels in the ISF. The device can also comprise a glucometer for discrete blood glucose measurements and can periodically measure glycosylayted hemoglobin with a HbA1c meter. The user of an infusion device with an HbA1c meter can be reminded every certain time period (e.g. 3 months) to check his/her Hb A1c level. In some embodiments, the HbA1c meter may only be applied if the user's RBC turn-over rate is normal (i.e. 120 days).

The insulin infusion device can alert the user if a blood vessel has been broken during insertion of the infusion set. Blood inside the infusion tubing may dilute the insulin delivered by the device and decrease its action. For example, if blood is detected in a significant amount (i.e. not a superficial capillary has been nicked but a more profound vessel has been broken), an alarm (e.g. audio, vibratory) can alert the user and the user can be instructed to remove the infusion set and insert a new one at another location. Also, if a subcutaneous hematoma is eventually created after insertion of the infusion set, the user can be instructed to remove the insertion set.

The means for alerting of unsuccessful insertion of the infusion set can comprise a reagent immobilized to the inside or outside of the cannula of the infusion set, wherein said reagent reacts with blood for rapid diagnostic detection of blood in the ISF. For example, the test can be based on the detection of hemoglobin, the peroxidase active component of blood.

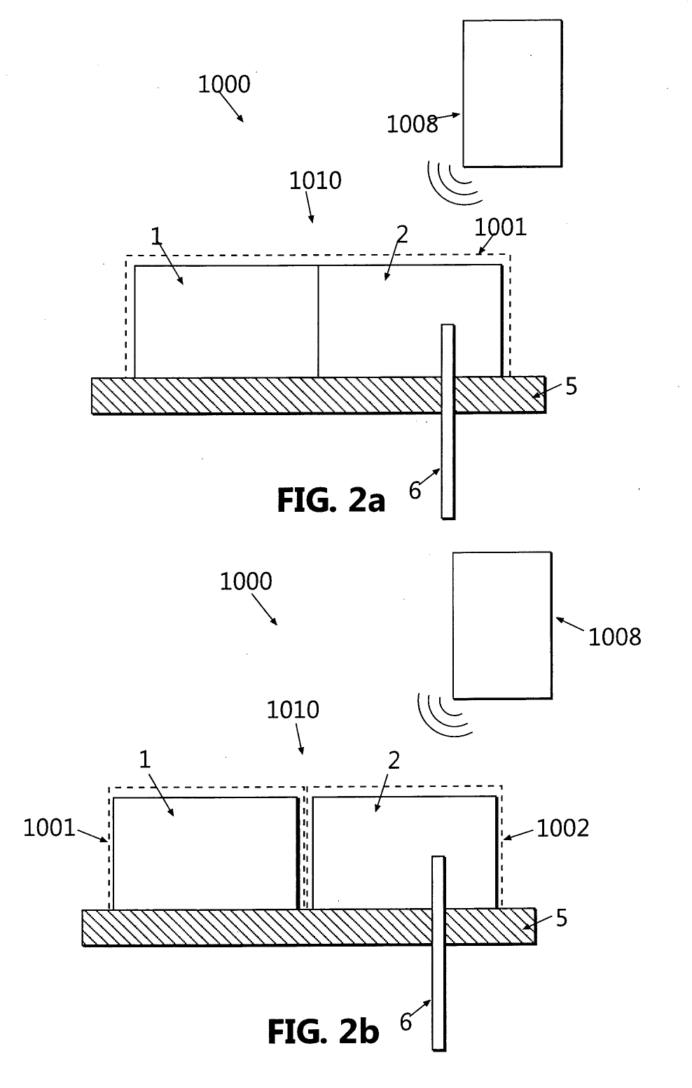

FIGS. 2a-b show one example of the device (1000) comprising an insulin dispensing patch unit (1010) adherable to the user's skin (5), and a remote control unit (1008) that can communicate with the patch unit (1010), allowing programming, user inputs and data acquisition.

The patch unit (1010) can be composed of one part with one housing (1001), as shown in FIG. 2a, or two parts with two housings (1001, 1002) comprising reusable (1) and disposable (2) parts respectively, as shown in FIG. 2b. The patch unit (1010) can be coupled to a cannula (6) that can penetrate the skin (5) to allow delivery of insulin.

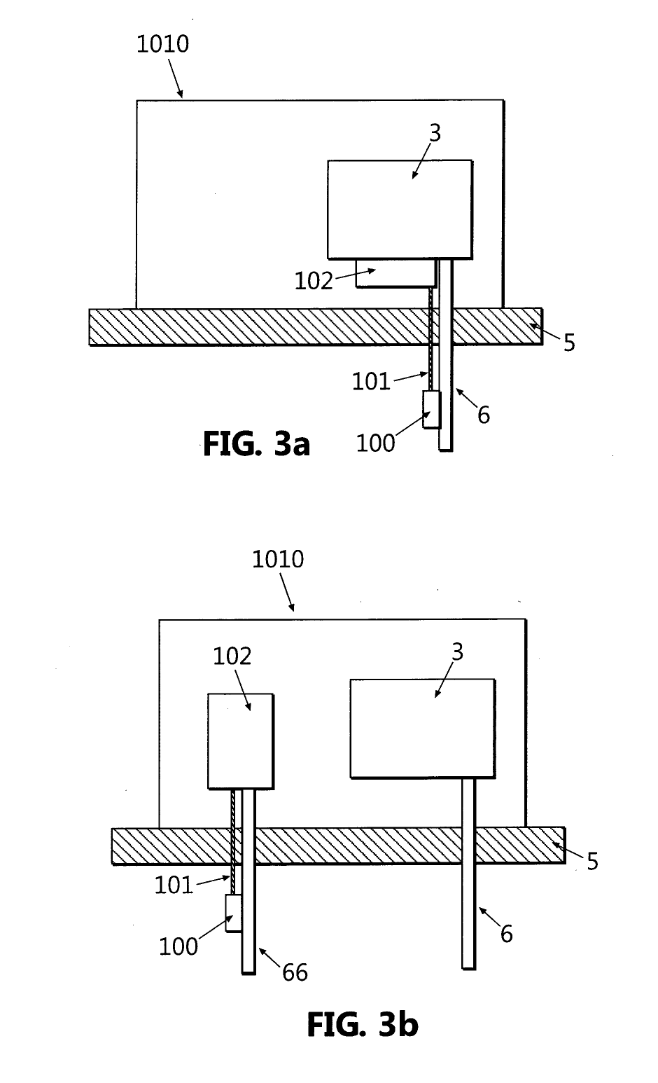

FIGS. 3a-b show an insulin dispensing patch (1010) that can include a subcutaneous ISF ketone management apparatus. The ISF ketone management apparatus can comprise a subcutaneous sensing means (100), electrical wiring (101), and a processing means (102).

In FIG. 3a, a single cannula (6) that can penetrate the skin (5) can be used for delivery of insulin from a reservoir (3) and for monitoring ketone levels in the ISF via a ketone sensing means (100).

In FIG. 3b, one cannula (6) can be used for delivery of insulin and the ISF ketone sensing means (100) can be located on a separate penetrating element (66).

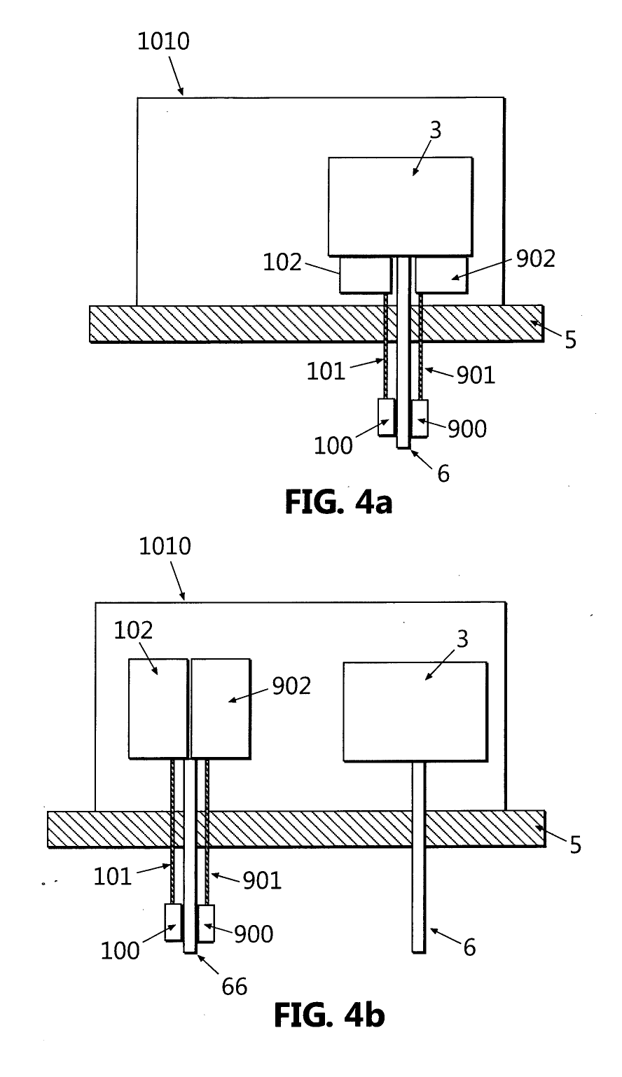

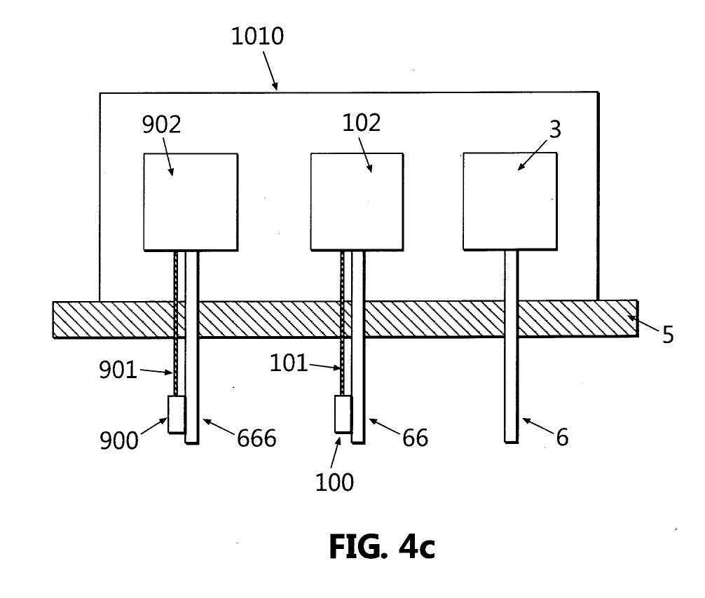

FIGS. 4a-c show some examples of the insulin dispensing patch (1010), the subcutaneous ISF glucose monitoring apparatus, and the subcutaneous ISF ketone management apparatus. The ISF glucose monitoring apparatus can comprise a subcutaneous sensing means (900), electrical wiring (901), and a processing means (902). The ISF ketone management apparatus can comprise a subcutaneous sensing means (100), electrical wiring (101), and a processing means (102).

In FIG. 4a, a single cannula (6) that penetrates the skin (5) is used for insulin delivery from a reservoir (3) and for monitoring ketone levels and glucose levels in the ISF.

In FIG. 4b, one cannula (6) can be used for insulin delivery and the ISF ketone sensing means (100) and ISF glucose sensing means (900) can be located on a designated element (66).

In FIG. 4c, one cannula (6) can be used for delivery of insulin, the ISF ketone sensing means (100) can be located on a designated element (66), and the ISF glucose sensing means (900) can be located on another designated element (666).

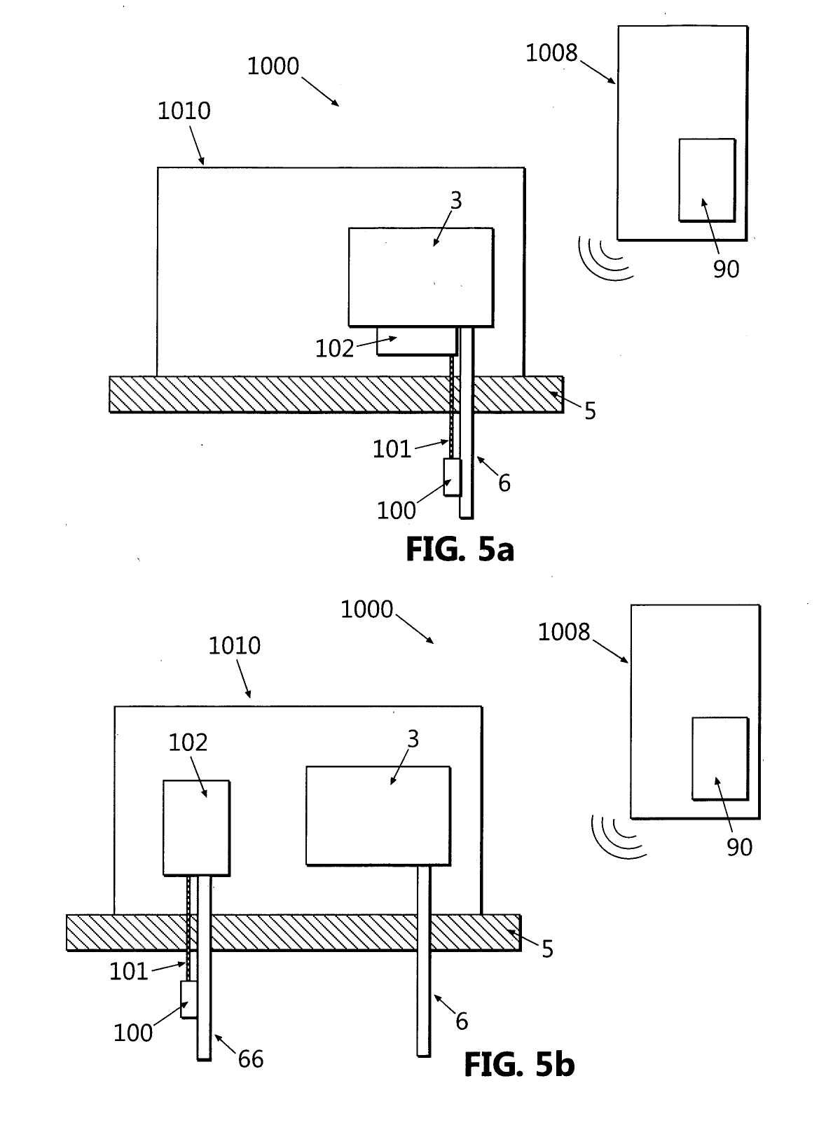

FIGS. 5a-b show a device (1000) comprising an insulin dispensing patch unit (1010) with a subcutaneous ISF ketone management apparatus, and a remote control unit (1008) comprising a blood glucose monitor (90) for discrete blood glucose measurements. The remote control unit (1008) can communicate with the patch unit (1010), allowing programming, user inputs and data acquisition. The ISF ketone management apparatus can comprises a subcutaneous sensing means (100), electrical wiring (101), and a processing means (102).

In FIG. 5a a single cannula (6) that penetrates the skin (5) can be used for delivery of insulin from a reservoir (3) and for monitoring ketone levels in the ISF.

In FIG. 5b insulin can be delivered via a cannula (6) and ketone management can be carried out by virtue of a designated element (66).

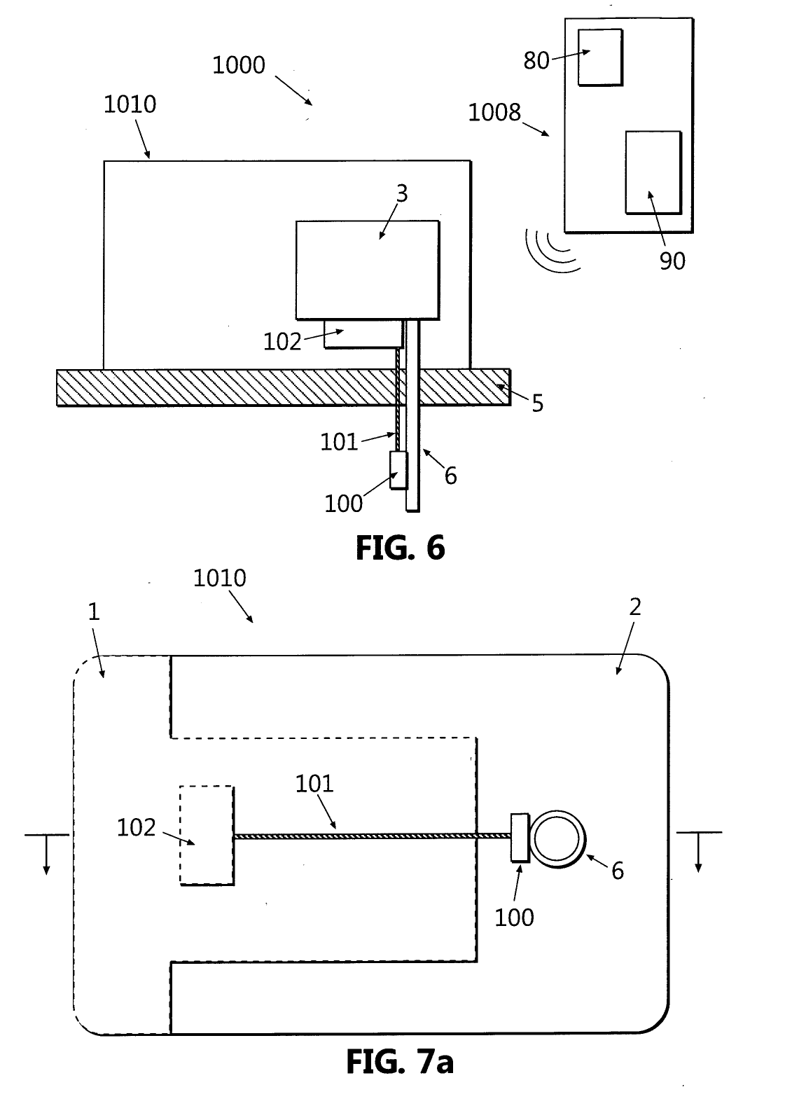

FIG. 6 shows a device (1000) comprising an insulin dispensing patch unit (1010) with a subcutaneous ISF ketone management apparatus, and a remote control unit (1008) that can contain a blood glucose monitor (90) and HbA1c monitor (80). A single cannula (6) that penetrates the skin (5) can be used for delivery of insulin from a reservoir (3) and for monitoring ketone levels in the ISF. The ISF ketone management apparatus can comprise a subcutaneous sensing means (100), electrical wiring (101), and a processing means (102). Alternatively (not shown) ketone sensing can be carried out by a virtue of a designated element different cannulae.

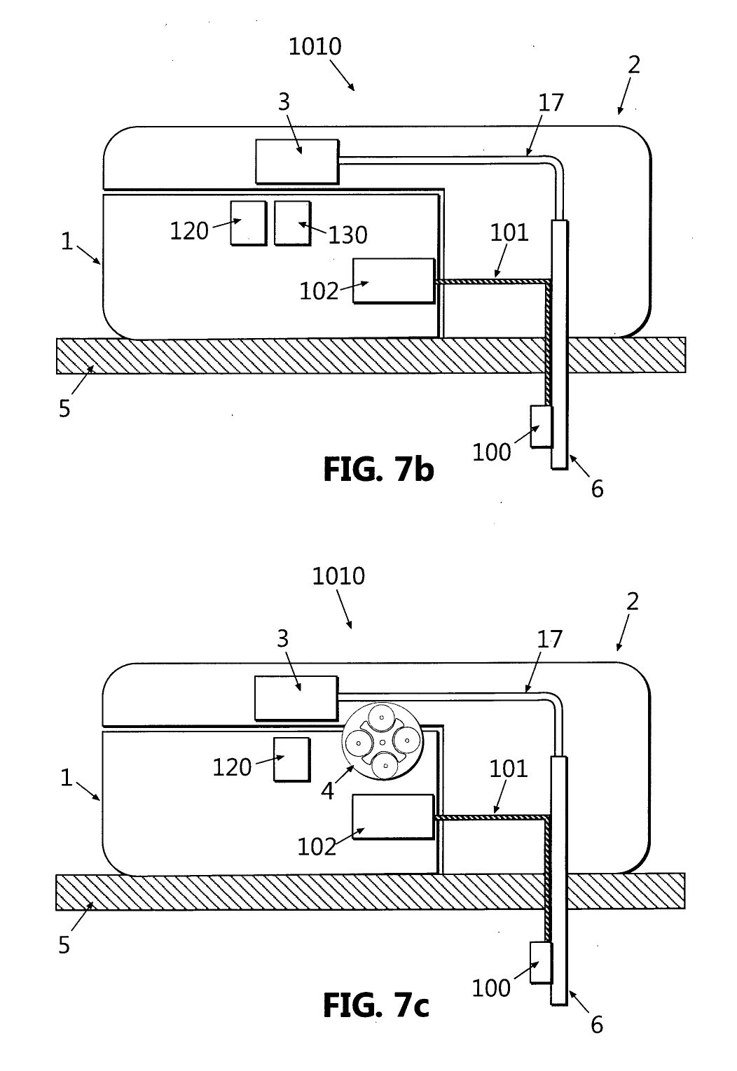

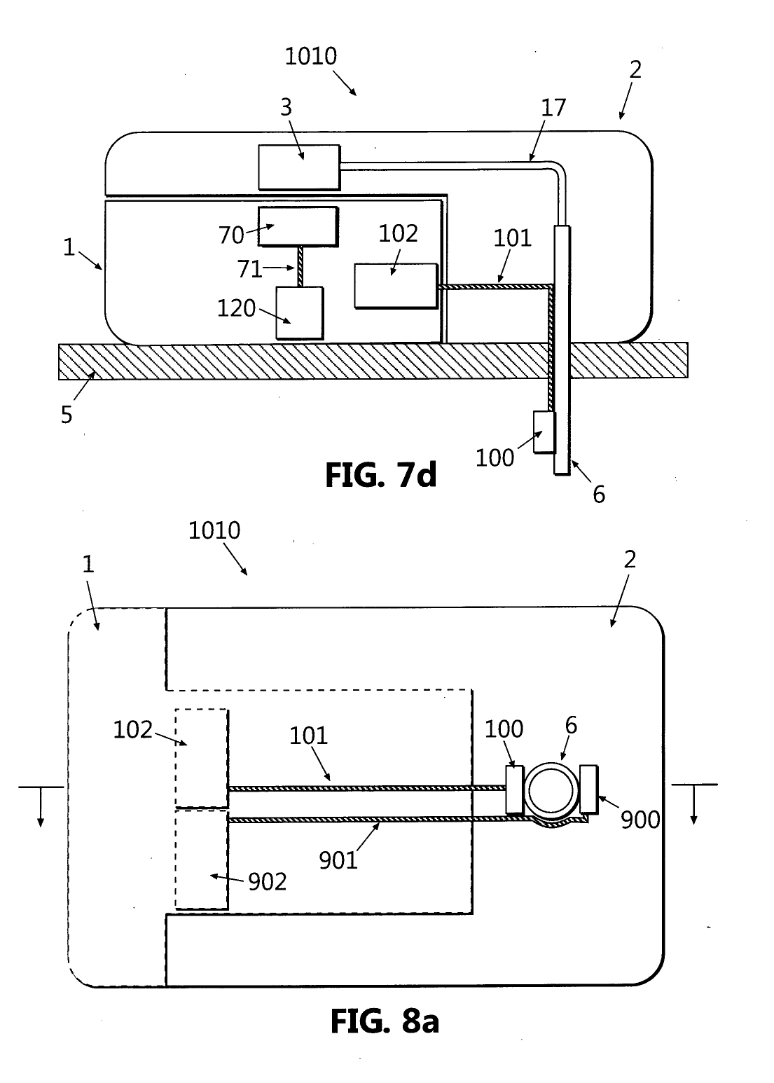

FIGS. 7a-d show two part insulin dispensing patch unit (1010) that can continuously infuse insulin and monitor ketone levels in the ISF. FIG. 7a shows a top view of the patch unit composed of a reusable part (1) and a disposable part (2). FIGS. 7b-d show a side view of the patch unit.

The two part insulin dispensing patch unit can comprise a disposable part (2) which can include the reservoir (3) and delivery tube (17) and can be coupled to a cannula (6). The reusable part (1) can comprise a driving mechanism (120) and a pumping mechanism (130). The pumping mechanism can be a peristaltic type (4), as shown in FIG. 7c or a piston/plunger type (“syringe type”), as shown in FIG. 7d. In a syringe type mechanism a rod (71) and a propelling plunger (70) can be used to displace fluid from a reservoir (3). The ISF ketone management apparatus can comprise a subcutaneous sensing means (100), electrical wiring (101), and processing means (102) located within the reusable part. The device can comprise a remote control unit (not shown) with an integrated blood glucose monitor.

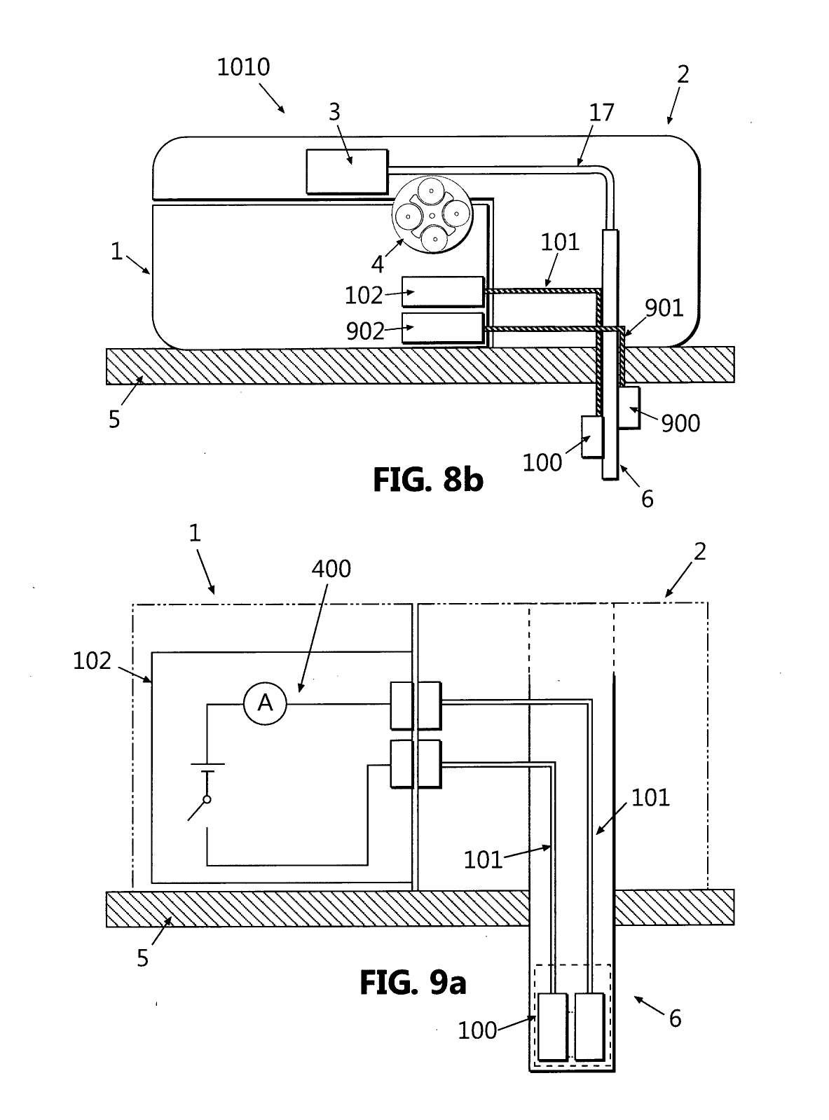

FIGS. 8a-b show a two part insulin dispensing patch unit (1010) that can continuously monitor glucose levels and ketone levels in the ISF. FIG. 8a shows a top view of the patch unit composed of a reusable part (1) and a disposable part (2). FIG. 8b shows a side view of the patch unit.

The two part insulin dispensing patch unit can comprise a disposable part (2) which can include a reservoir (3), a delivery tube (17) and can be coupled to a cannula (6). The reusable part (1) can include a pumping mechanism (e.g. peristaltic). The ISF ketone and ISF glucose monitoring apparatuses can comprise each a subcutaneous sensing means (100, 900, respectively), electrical wiring (101, 901), located within the disposable part (2), and a processing means (102, 902, respectively) located within the reusable part (1).

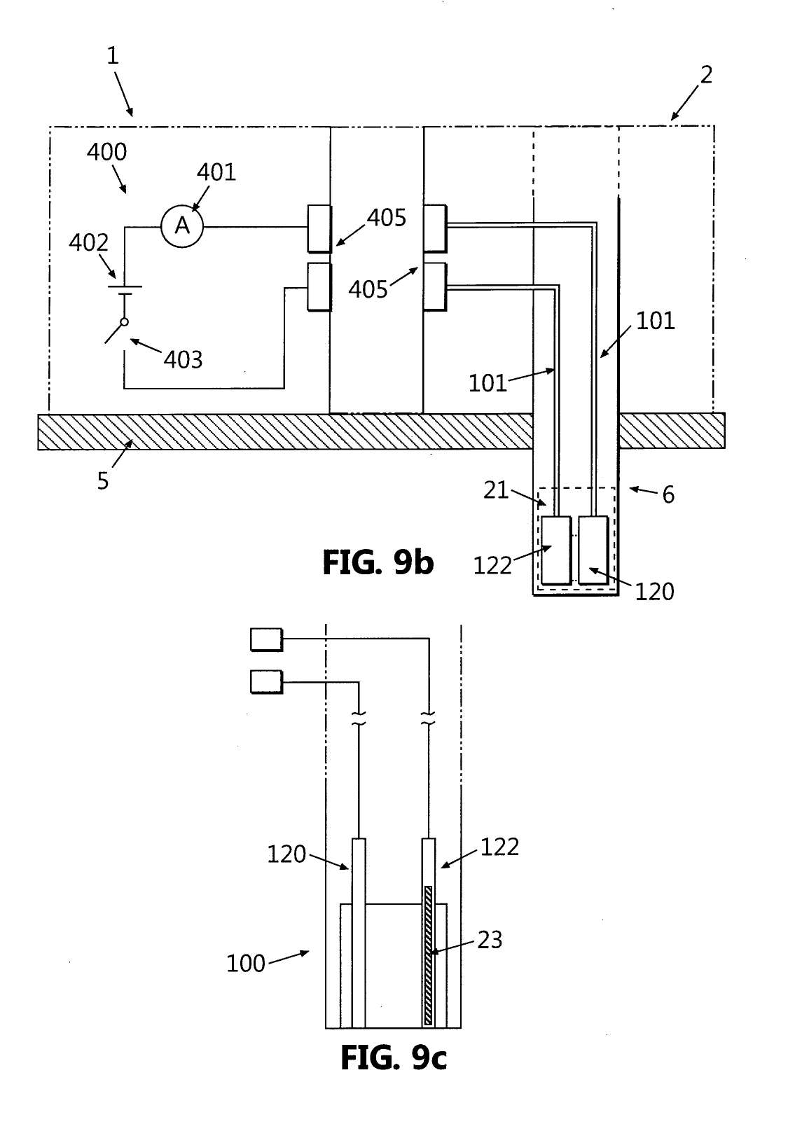

FIGS. 9a-c provide some examples of an ISF ketone management apparatus that can be shared by the two parts of the patch unit. FIG. 9a shows the paired two part patch with the reusable part (1) connected to the disposable part (2). FIG. 9b shows the two parts disconnected.

The ketone sensing means (100) can be located in the disposable part (2) and the sensor processing means (102) can be located in the reusable part (1). The sensor processing means can comprise an amperometric circuit (400). Once the reusable part (1) and the disposable part (2) are connected, the amperometric circuit can be electrically closed, as shown in FIG. 8a.

FIG. 9b shows the reusable part provided with an amperometric circuit (400) comprising amperometer (401), battery (402), switch (403), and contact means (405). The disposable part provided with electrodes (122,120), electrically insulating layer (21), and wiring (101) electrically connected to contact means (405) is also shown. The electrodes are located on the outside portion of the cannula (6) preferably on the subcutaneous portion. When the amperometric circuit is closed, electrons flow from the electrodes (122) towards the positive pole of battery (402) and the amperometer (401) can measure the flowing current. The switch (403) controls circuit operation for energy saving. In some embodiments, various component of the electrical circuit can reside in the reusable and disposable part interchangeably, for example, the battery can reside in the disposable part.

FIG. 9c shows in detail the electrodes of the ketone sensing means (100). The sensing means contains at least 2 electrodes—at least one working electrode, at least one counter electrode and optionally a reference electrode. The electrochemical reaction (for example a reaction catalyzed by the enzyme 3-hydroxybutyrate dehydrogenase (HBDH) for the detection of ketone bodies) takes place on the working electrode (122) which can be covered by a sensing layer (23). The sensing layer (23) can comprise at least the catalytic enzyme and, preferably, 3-hydroxybutyrate dehydrogenase (HBDH). The analyte sensing layer (23) can be immobilized on the working electrode (122) by any methods known in the art (e.g. cross-linking, entrapment). A counter electrode (120) can be paired with the working electrode (122), thereby allowing electrical circuit closing. A reference electrode can be used for measuring the working electrode potential.

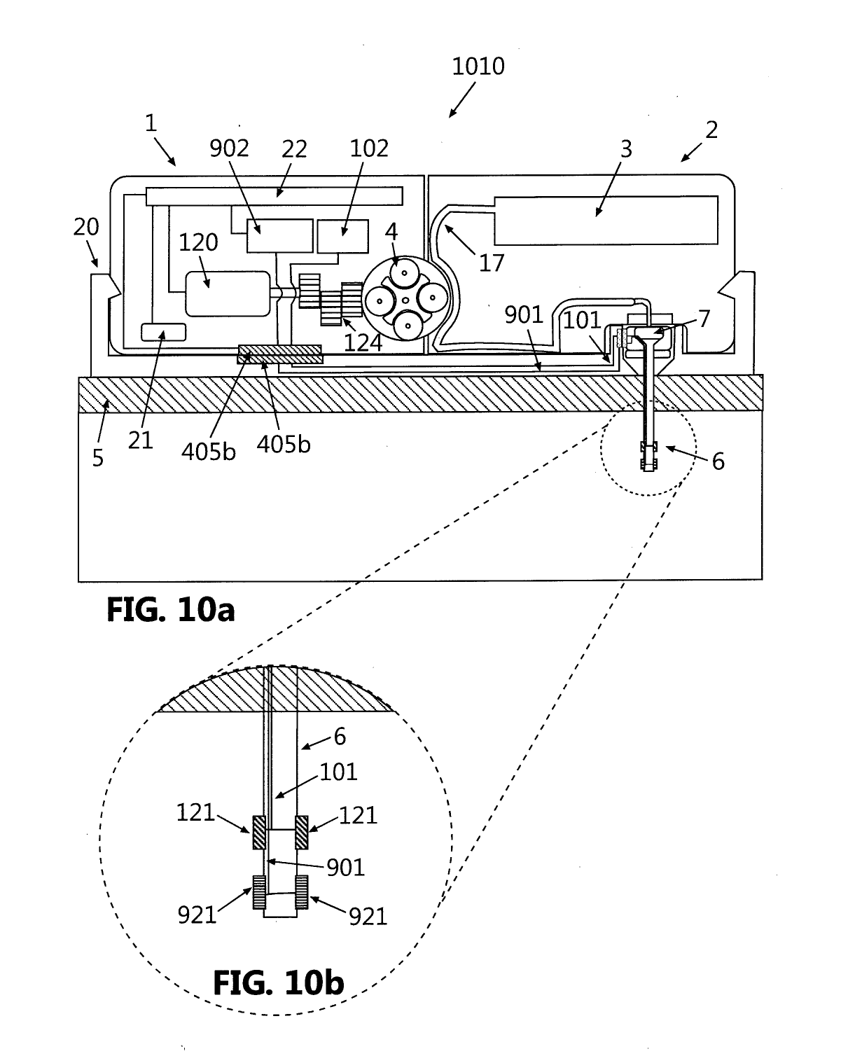

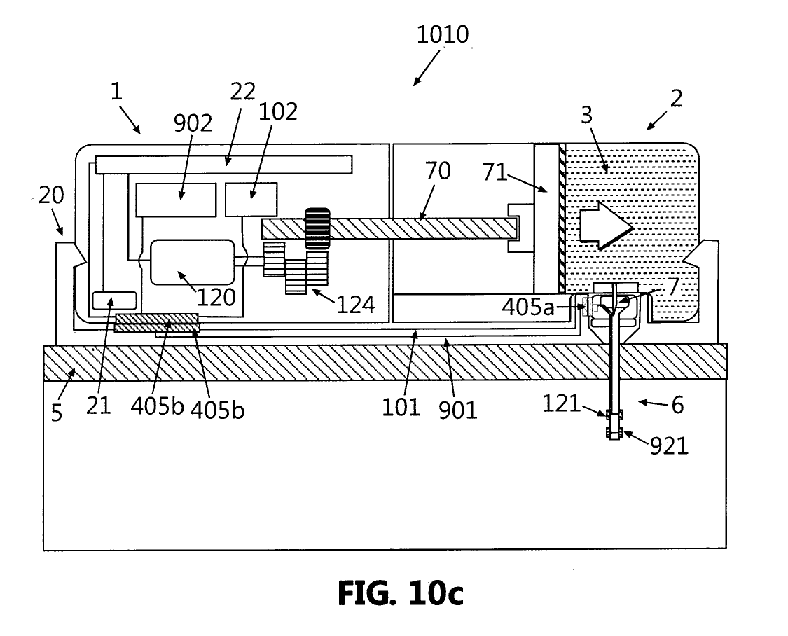

FIGS. 10a-c show some embodiments of the insulin dispensing patch unit (1010) that can dispense insulin and continuously monitor ISF glucose and ISF ketone levels and can be remotely controlled. In FIG. 10a the pumping mechanism is peristaltic. In FIG. 10c the pumping mechanism is of a syringe type. The dispensing patch unit (1010) can be composed of a disposable part (2) and a reusable part (1). Each part may be contained in a separate housing (not shown). The disposable part (2) can contain a reservoir (3) and an outlet port (7) that can be connected and disconnected to a cannula (6). The reusable part (1) can contain electronics (e.g. PCB, processor, etc) (22), driving mechanism (120), pumping mechanism, and an energy source (21). Some of these elements (e.g. the energy source (21)) can be located in the disposable part. Some elements required for the ISF glucose and ISF ketone management apparatuses (902, 102, respectively) can be located either in the disposable or in the reusable parts.

FIG. 10a provides one example of the peristaltic pumping mechanism. A rotary wheel (4) contained in the reusable part (1) can positively displace fluid from the reservoir (3) by sequentially squeezing a delivery tube (17) provided in the disposable part.

FIG. 10c provides one example of a syringe type pumping mechanism. A pumping mechanism (120) connected to a gear, plunger rod (70), and plunger (71) can displace fluid from a reservoir (3) that can be contained in the disposable part (2).

A cradle unit (20) can be configured as a flat sheet that adheres to the skin (5). The cradle unit (20) can allow patch (1010) disconnection and reconnection upon patient's discretion. A cannula (6) can be inserted into the subcutaneous compartment through a dedicated passageway in the cradle unit. Electrodes covered with immobilized enzymes for the detection of ISF ketones and glucose (121, 921 respectively) can be attached to the outer surface of the cannula (6) as can be seen in enlargement in FIG. 10b. The electrons generated by the enzymatic reaction can be accepted by the electrodes and the current can be delivered by wires to connectors located on the upper portion of the cannula (405a). Electrical wires (101, 901) can electrically connect cannula-cradle and cradle-patch unit connectors (405a and 405b respectively).

After connection of the patch (1010) to the cradle (20), connectors in the cradle touch connectors in the reusable part of the patch unit (405b) and current is delivered to the patch processors (102, 902) to be analyzed and interpreted for analyte concentration (i.e. glucose, ketones).

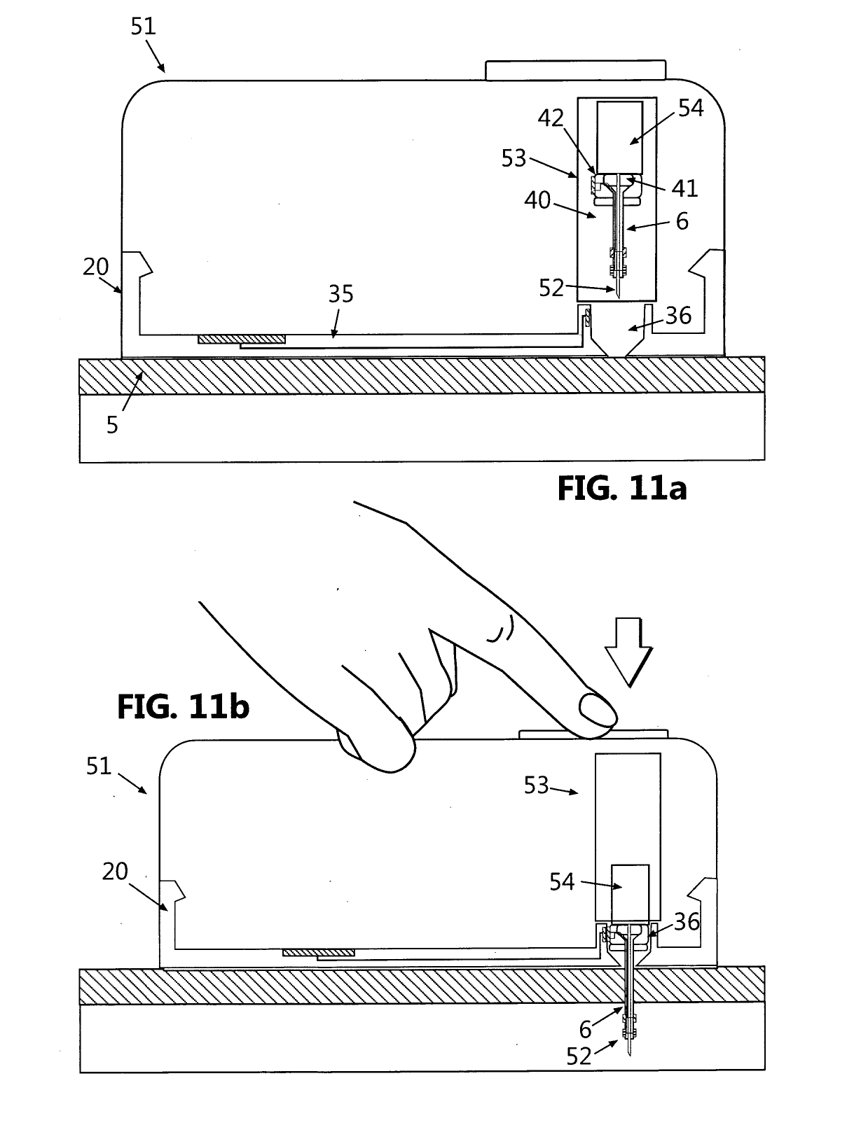

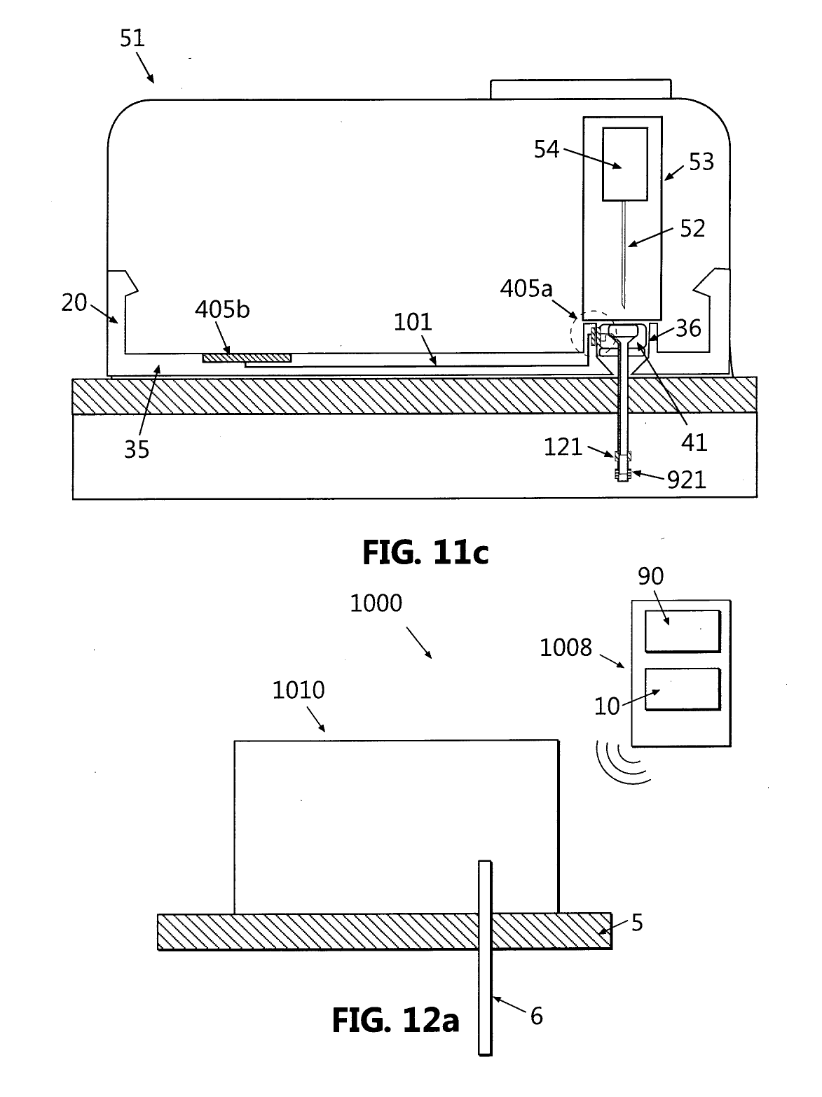

FIGS. 11a-c show one example of an automatic cannula insertion as detailed in our previous co-owned/co-pending Provisional Patent Application No. 60/937,155, filed Jun. 25, 2007, and titled “Insertion Device for Inserting a Cannula into a Body”, incorporated herein by reference in its entirety. A spring loaded inserter fires the cannula through the cradle passageway (well) (36) into the body and consequently the sharp penetrating member (52) and grip (54) is retracted into a protective shield (“protector”) (53). After cannula insertion, the connectors of the sensing apparatus can be properly assembled.

FIG. 11a shows one example of a cross-sectional view of the inserter (51) connected to the cradle unit (20) which can comprise a cradle base (35) and a well (36). The cannula cartridge unit (40) can include the protector (53), the penetrating member (52), the grip (54), the cannula (6), the cannula hub (41), and the rubber septum (42). FIG. 11b shows the cannula (6) within the body after firing. The penetrating member (52) is still in the subcutaneous compartment. FIG. 11c shows the final stage of cannula insertion. The cannula (6) is in the subcutaneous tissue, penetrating member (52) is retracted and located within protector (53), and cannula hub (41) is rigidly connected to the well (36).

The outer surface of the cannula (6) can include the electrodes which can be covered with immobilized enzymes for the detection of ISF ketones and/or ISF glucose (121, 921 respectively). Current can be flowing from the subcutaneous electrodes (121, 921) to connectors (405a) located on the upper portion of the cannula hub (41) and through wiring (101) to connectors (405b).

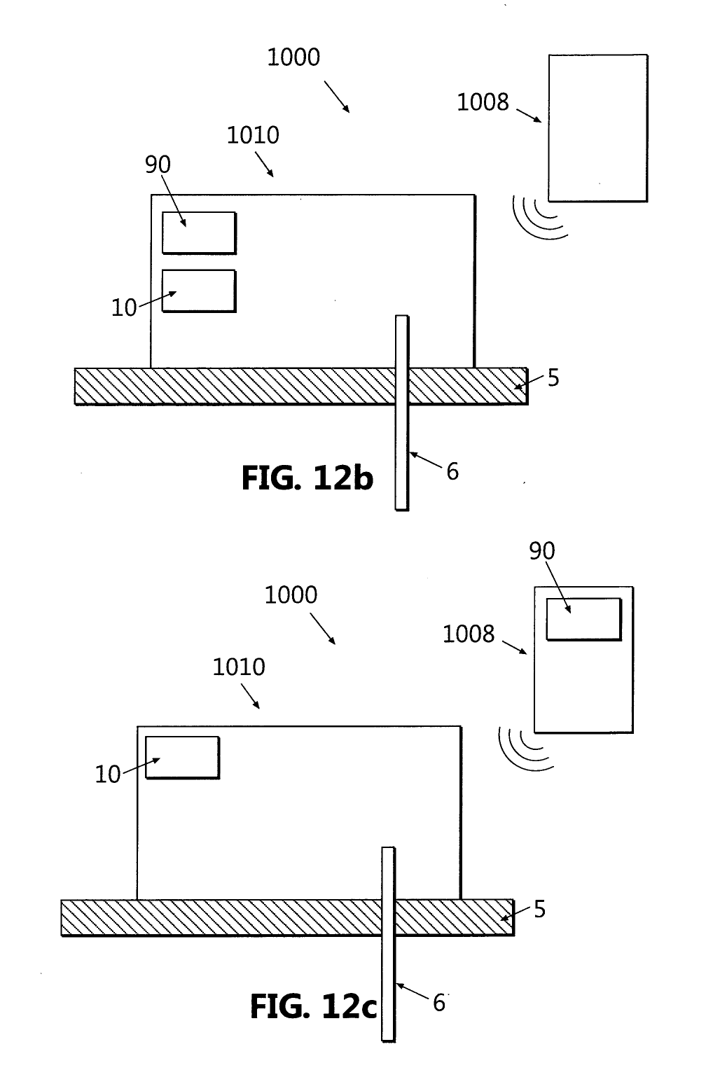

FIGS. 12a-c show a device (1000) that can comprise an insulin dispensing patch unit (1010) and a remote control unit (1008). The remote control unit (1008) can employ blood glucose and blood ketone monitors for discrete measurements (90, 10, respectively), carried out upon user discretion. The insulin dispensing patch (1010) can be coupled to a cannula (6) that can penetrate the skin (5) to allow delivery of insulin. The remote control unit (1008) can communicate with the patch unit (1010), allowing programming, user inputs and data acquisition.

In FIG. 12a, the blood glucose and blood ketone monitors (90, 10) are located in the remote control unit (1008). In FIG. 12b, the blood glucose and blood ketone monitors (90, 10) are located within the insulin dispensing patch unit (1010). In FIG. 12c, the blood glucose monitor (90) is located within the remote control unit (1008) and the blood ketone monitor (10) is located within the insulin dispensing patch unit (1010). According to another embodiment (not shown), the blood glucose monitor is located in the insulin dispensing patch unit and the blood ketone monitor can be located in the remote control unit.

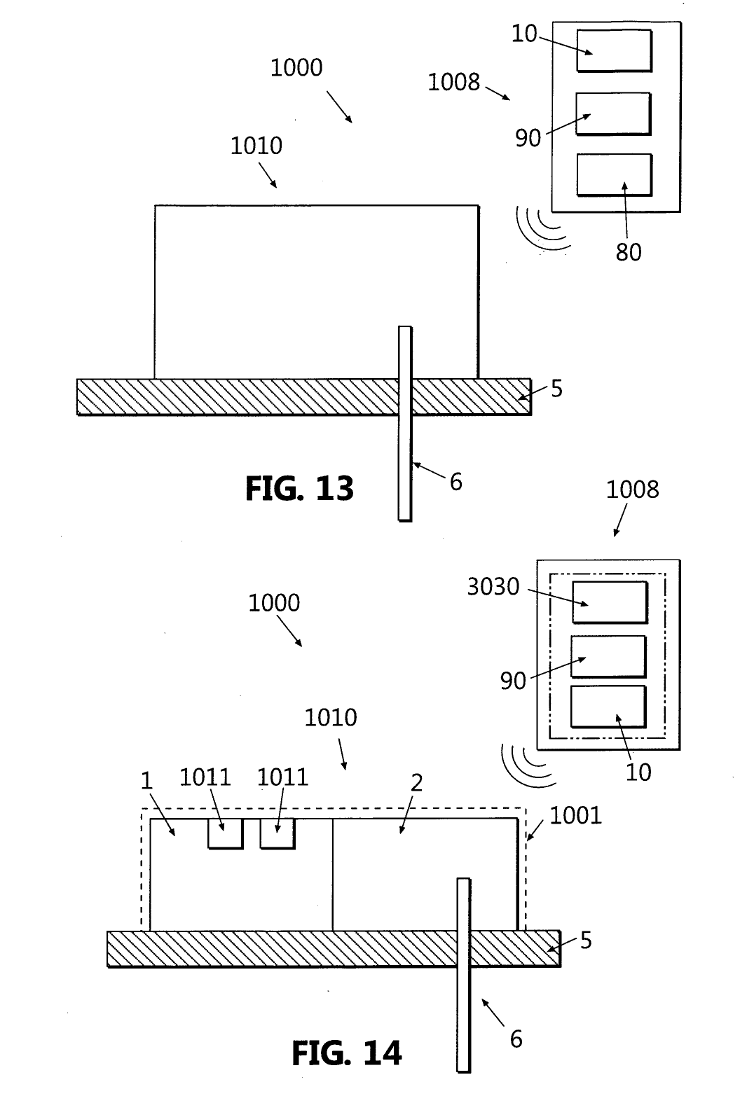

FIG. 13 shows a device (1000) comprising an insulin dispensing patch unit (1010) and a remote control unit (1008) that contain a blood glucose, ketone and HbA1c monitors (90, 10, 80, respectively). The insulin dispensing patch unit (1010) can be coupled to a cannula (6) that penetrates the skin (5) to allow delivery of insulin. The remote control unit (1008) can communicate with the patch unit (1010), allowing programming, user inputs and data acquisition. According to other embodiments (not shown), the blood glucose, ketone and HbA1c monitors (90, 10, 80, respectively) can be divided between the dispensing patch unit (1010) and the remote control unit (1008).

FIG. 14 shows the device (1000) comprising insulin dispensing patch unit (1010), which can be adherable to the user's skin (5), and a remote control unit (1008). Manual inputs can be carried out by buttons (1011) located on the patch unit (1010). The patch unit (1010) can be composed of one housing (1001) or two housings comprising reusable (1) and disposable (2) parts. The patch unit (1010) can be coupled to a cannula (6) that penetrates the skin (5) to allow delivery of insulin.

The remote control unit (1008) can have a display (3030) and can contain a blood glucose monitor (90) and a blood ketone monitor (10) for discrete glucose and ketone measurements respectively. An HbA1C monitor can be positioned in the reusable part of the patch unit or in the remote control unit.

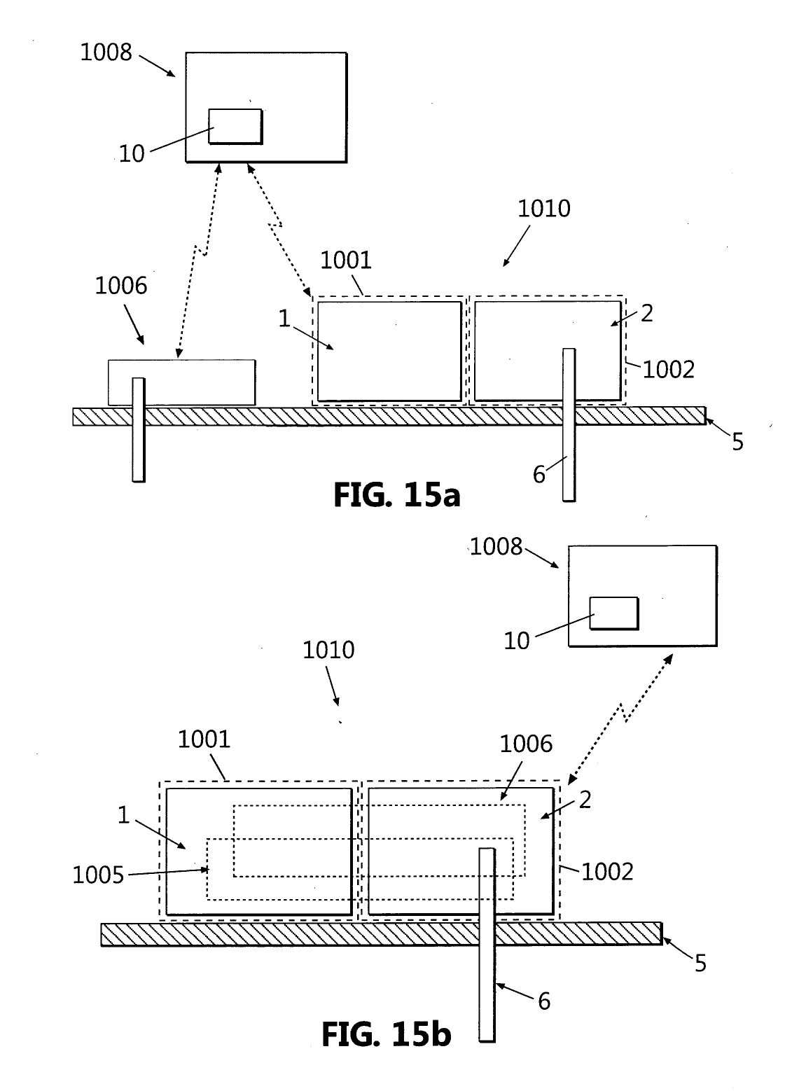

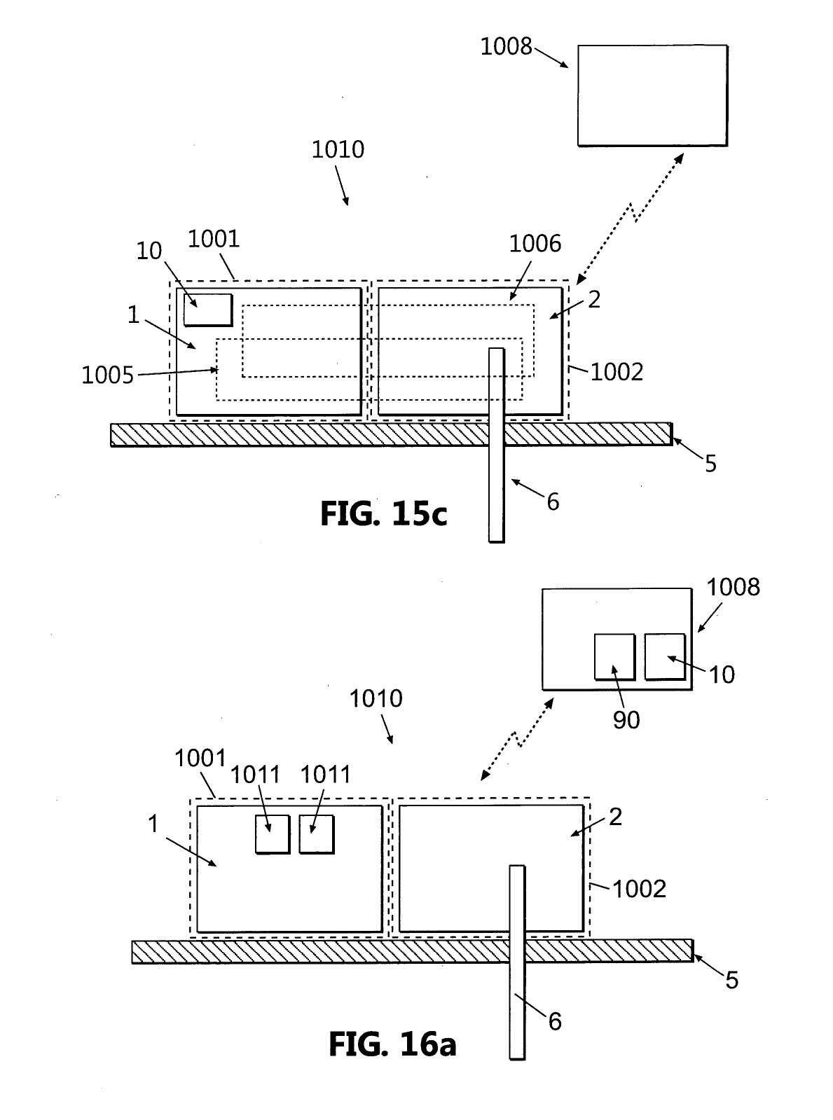

FIGS. 15a-c illustrate various configurations, in which blood glucose readings can be received from a continuous subcutaneous glucose monitor (1006). A communication channel between the continuous subcutaneous glucose monitor (1006) and a ketone monitor (10) for discrete blood ketone measurements can be maintained, allowing programming, data handling, and user inputs. For example, in FIGS. 15a-b the ketone monitor (10) for discrete blood ketone measurements resides in the remote control unit (1008).

FIG. 15a illustrates an embodiment in which ISF glucose can be measured by a separate continuous subcutaneous glucose monitor (1006). FIG. 15b-c illustrates embodiments in which the continuous subcutaneous ISF glucose sensing (monitoring) apparatus (1006) can be integrated within the patch unit (1010) of the device.

The insulin dispensing apparatus (1005) and glucose sensing apparatus (1006) can constitute a single dispensing patch unit, and can use a single cannula (6) for both dispensing and sensing as described in detail in our previous U.S. application Ser. No. 11/706,606 (herein incorporated by reference in its entirety).

The sensing apparatus and the dispensing apparatus can have separate cannulae that can penetrate the skin (5) and reside in the subcutaneous tissue. The dispensing patch unit of this embodiment can be comprised of two parts—a reusable part (1) and a disposable part (2), each part has a corresponding housing (1001, 1002). According to another embodiment, as shown in FIG. 15c, a blood ketone monitor can be integrated in the sensing and dispensing patch unit.

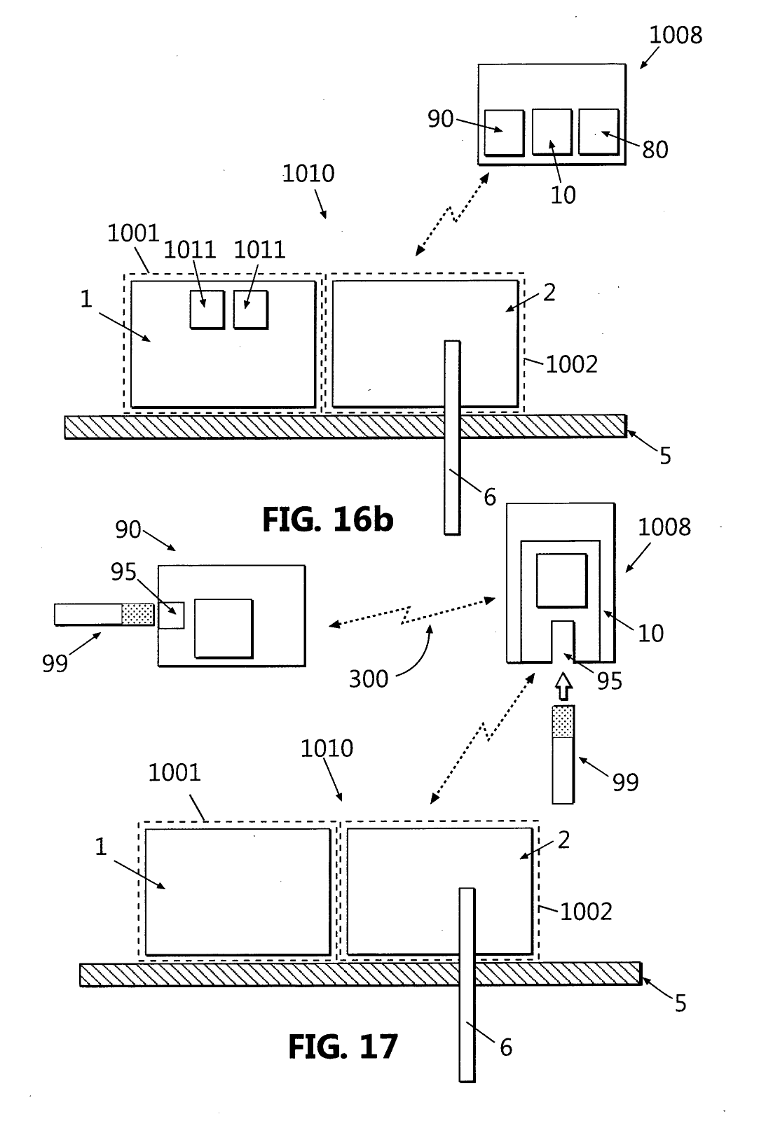

FIGS. 16a-b illustrate one embodiment of a patch unit (1010) with two parts located in two housings (1001, 1002)—a reusable part (1) and a disposable part (2). The relatively cheap components of the patch unit reside in the disposable part (2) and the relatively expensive components reside in the reusable part (1). In another preferred embodiment (not shown) the patch unit can be attachable to a skin adhered cradle unit allowing the connection and disconnection of the patch unit to and from the body.

The device can comprise a remote control unit (1008) with an integrated blood ketone monitor (10) and blood glucose monitor (90), as shown in FIG. 16a. Programming can be carried out by the remote control or by buttons (1011) located on the patch unit. According to one embodiment, as shown in FIG. 16b, the device can also comprise an HbA1c monitor (80) located in the remote control unit.

FIG. 17 shows one embodiment of the device in which a blood ketone monitor (90) can be located in the remote control unit (1008) and blood glucose readings can be directly or remotely received from an independent monitor (90).

The blood glucose monitor (90) and the blood ketone monitor (10) can each comprise an opening (95) in the housing for receiving a test strip (99). The user can extract blood from the body, place a blood drop on the test strip (99) and insert the strip (99) into the opening (95). The readings can be displayed on a screen. A communication channel (300) between the blood glucose monitor (90) and the remote control unit (1008) can be maintained.

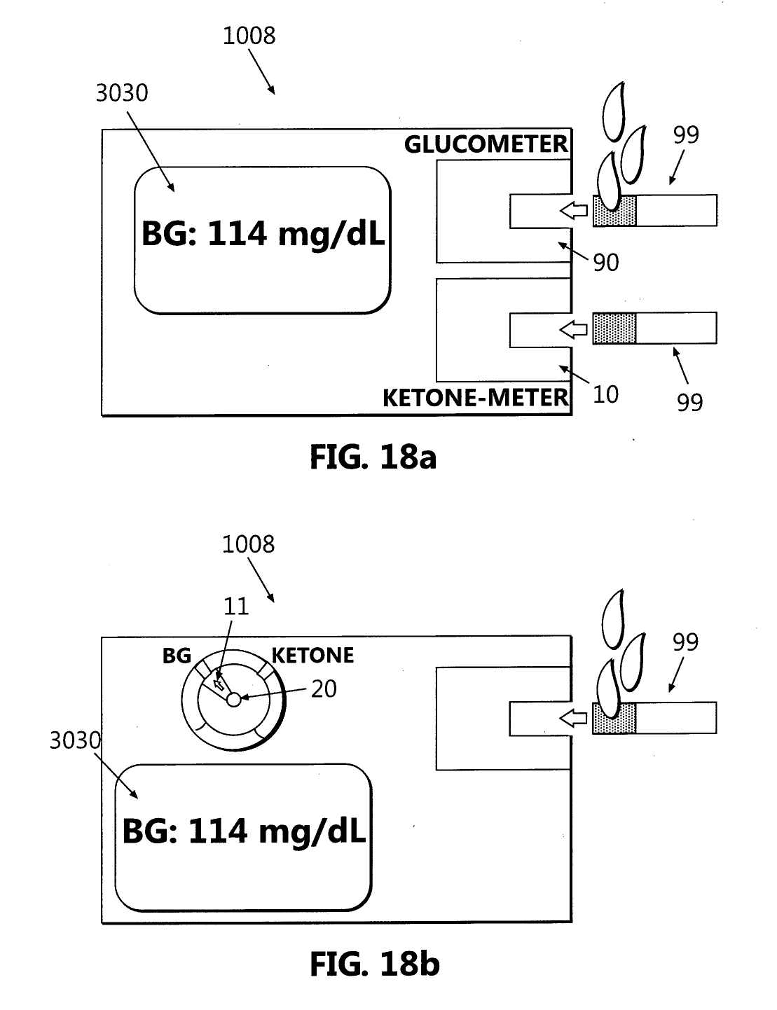

FIGS. 18a-b show two embodiments of a remote control unit (1008) comprising a blood glucose (BG) monitor (90) and a blood ketone monitor (10). In FIG. 18a, each monitor can comprise an independent opening (95) for receiving a test strip (99). The readings can be displayed on a common screen (3030).

In FIG. 18b, the BG monitor and the blood ketone monitor can share an opening (95) for receiving of a test strip (99). The readings are displayed on a screen (3030). The remote control unit can comprise a button (20) with a pointer (11). In some embodiments, the user can check BG or Ketone by rotating the pointer to “BG” or “Ketones” and pressing the button.

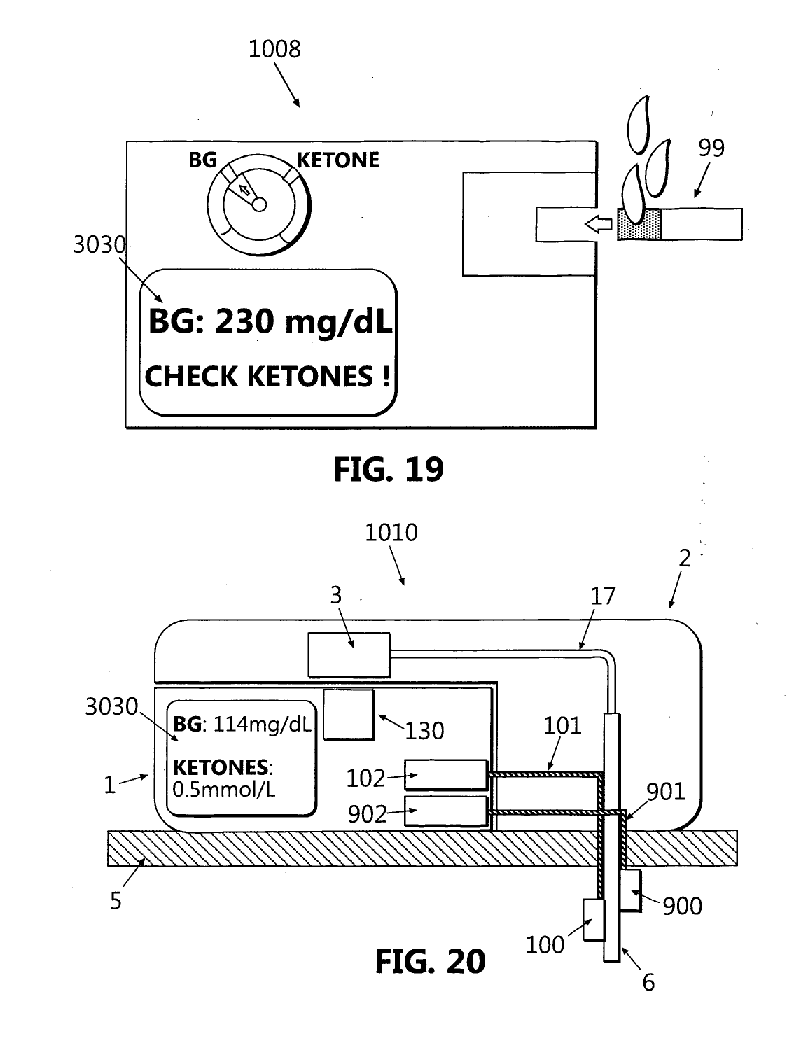

FIG. 19 shows the remote control unit (1008) comprising BG and Ketone monitors with a common display (3030). For example, if the user's blood glucose is high, a “check ketones” warning can be displayed.

FIG. 20 shows a device comprising a two part patch unit (1010) with a subcutaneous ISF glucose sensing apparatus, a subcutaneous ISF ketone sensing apparatus and a display screen located on the reusable part of the patch unit. The insulin dispensing patch unit (1010) can comprise a disposable part (2) which includes insulin reservoir (3), delivery tube (17) and can be coupled to a cannula (6). The reusable part can include a pumping mechanism (130) and display screen (3030). The ketone and glucose monitoring apparatuses comprise each a subcutaneous sensing means (100, 900 respectively), electrical wiring (101, 901), located within the disposable part (2), and a processing element (102,902) located within the reusable part (1).

Any and all patents, applications, articles and/or publications referenced in this specification are hereby incorporated by reference herein in their entireties. Although particular embodiments have been disclosed herein in detail, this has been done by way of example for purposes of illustration only, and is not intended to be limiting with respect to the scope of the appended claims, which follow. Other embodiments, advantages, and modifications are considered to be within the scope of the following claims.

What is claimed is:

1.-46. (canceled)

47. A ketone management device comprising:
at least one subcutaneously insertable element, the element having a proximal portion and a distal portion, the proximal portion coupled to a housing, the distal portion configured for subcutaneous placement within a human body;
a plurality of electrodes coupled to the distal portion of the at least one subcutaneously insertable element;
at least one of the plurality of electrodes being covered by an immobilized enzyme 3-hydroxybutyrate dehydrogenase (HBDH); and
a processor adapted to correlate a signal received from the plurality of electrodes to a concentration of ketone in the human body.

48. The device of claim 47, wherein the processor is wirelessly connected to the plurality of electrodes.

49. The device of claim 47, wherein the processor is additionally adapted to correlate the signal received from the plurality of electrodes to a concentration of glucose in the human body.

50. The device of claim 47, wherein the plurality of electrodes comprises a first electrode having a first exposed surface that is at least partially coated with the immobilized enzyme 3-HBDH and a second electrode having a second exposed surface that is substantially free of the electrochemical reagent.

51. The device of claim 50, wherein the enzyme 3-HBDH undergoes a chemical reaction with analyte bodies, the chemical reaction producing a measurable voltage or current differential between the first electrode and the second electrode.

52. The device of claim 50, wherein the processor is provided in a remote control.

53. The device of claim 52, wherein the remote control unit comprises an HbA1c measurement device.

54. The device of claim 47, wherein the processor is configured to generate a notification of impending diabetic ketoacidosis based on the signals received from the plurality of electrodes.

55. The device of claim 47, wherein the device is adapted for continuous monitoring of the concentration of ketone in a subcutaneous compartment of the human body.

56. The device of claim 55, wherein the continuous monitoring is accomplished by performing discrete measurements via at least one of a high measurement rate and short inter-measurement intervals.

57. The device of claim 47, wherein the at least one subcutaneously insertable element is configured for monitoring ketone concentration levels in the subcutaneous tissue and another subcutaneously insertable element is configured for continuously dispensing insulin to the subcutaneous tissue.

58. The device of claim 50, wherein the concentration of ketone in the human body is determined based on an enzymatic dehydrogenation to acetoacetate by 3-hydroxybutyrate dehydrogenase (HBDH), followed by the detection of the reduced form of nicotinamide adenine (NADH).

59. The device of claim 58, wherein the generated NADH is further recycled to NAD+ by salicyclate hydroxylase (SHL).

60. The device of claim 47, wherein the at least one subcutaneously insertable element includes glucose oxidase (GOX).

61. The device of claim 47, wherein the at least one of the plurality of electrodes comprising a plurality of layers that modulate the diffusion of multiple analytes in the subcutaneous tissue.

62. The device of claim 47, wherein the at least one subcutaneously insertable element comprises a single cannula configured for both monitoring ketone levels in the subcutaneous tissue and dispensing insulin into the subcutaneous tissue.

63. The device of claim 47, further comprising a pump.

64. The device of claim 47, further comprising a means for alerting of unsuccessful insertion of an infusion set of an insulin pump.

65. The device of claim 64, wherein the means for alerting of unsuccessful insertion of the infusion set operates upon reaction of the immobilized enzyme with blood in the ISF.

66. The device of claim 64, wherein the infusion set includes the at least subcutaneously insertable element, the at least one subcutaneously insertable element further includes the mean for alerting of unsuccessful insertion thereon.
